(19) United States
(12) Patent Application Publication
(10) Pub. No.: US 2025/0269126 A1
(43) Pub. Date: Aug. 28, 2025

(54) Methods of Improving Cognition

(71) Applicant: Alex Stenzler, Long Beach, CA (US)

(72) Inventor: Alex Stenzler, Long Beach, CA (US)

(21) Appl. No.: 19/059,499

(22) Filed: Feb. 21, 2025

Related U.S. Application Data
(60) Provisional application No. 63/557,225, filed on Feb. 23, 2024.

(51) Int. Cl. A61M 16/00; A61B 5/00

(52) U.S. Cl. CPC A61M 16/0045; A61B 5/4088

(57) ABSTRACT

The invention is based, at least in part, on the discovery that inhaling air having an elevated carbon dioxide level or concentration leads to improvements in cognition in subjects experiencing cognitive dysfunction or decline associated with a variety of diseases, disorders, and conditions.

### CROSS REFERENCE TO RELATED APPLICATIONS

This application claims priority to U.S. Provisional Application No. 63/557,225, filed Feb. 23, 2024, which is hereby incorporated by reference herein in its entirety.

### BACKGROUND OF THE INVENTION

During the worldwide COVID-19 pandemic, a significant population of patients reported different functional complaints one month or later after recovery from the acute infection. This entity has a number of names including “long COVID” or “post COVID condition” (PCC) or “post-COVID syndrome” (PCS). More than 30% of individuals affected by COVID-19 (Tenforde, et al., 2020, MMWR Morb Mortal Wkly Rep 69:993-998), including asymptomatic cases (Huang, et al., 2021, The Lancet 10302:747-758), and approximately 80% of patients hospitalized for COVID-19 may experience post-COVID sequelae. Fatigue and cognitive impairment, along with other enduring neuropsychiatric (e.g., depression) (Renaud-Charest, et al., 2021, J Psych Res 144:129-137) and physical (e.g., dyspnea) manifestations, comprise “post-acute sequelae of COVID-19” (i.e., symptoms persisting for at least 4 weeks following infection) (Nalbandian, et al., 2021, Nat Med 27:601-615), colloquially referred to as “long COVID” (Alwan and Johnson, 2021, Med (NY) 14:501-504). The National Institute for Health and Care Excellence (NICE) defines PCS as a constellation of symptoms which develop during or following COVID-19 infection, persist for >12 weeks, and are not sufficiently explained by alternative diagnoses.

Long-COVID is on the rise and no effective treatment exists yet to improve cognitive function (Sabel, et al., Restorative Neurology and Neuroscience. 2021; 39:393-408). Recent research has shown that people with even mild COVID had a greater decline in cognitive function, notably in their ability to perform complex tasks (Douaud, et al., 2022, Nature 604:697-707). These symptoms were associated with changes observed on MRI exams. In a U.K. Biobank study, 401 COVID patients showed a greater loss of gray matter volume and more brain tissue damage, an average of 4.5 months after infection, compared with people who never were infected. A key aspect of this study was its focus on people with mild COVID-19. Only 4% of patients were hospitalized during their bout of acute COVID-19, so the findings come from a population that parallels the experience of most people worldwide who have been infected.

Ceban performed a meta-analysis of 81 studies where a primary outcome indicated that 22% of individuals exhibited cognitive impairment 12 or more weeks following COVID-19 diagnosis (Ceban at al. 2022, Brain Behavior Immunity 101:93-135). Studies which objectively assessed cognitive impairment reported significantly greater proportions of individuals with cognitive impairment as compared to those employing subjective modes of ascertainment. There was no statistically significant difference between hospitalized and non-hospitalized subgroup proportions reporting post-COVID cognitive impairment.

Multiple studies have identified neuroanatomical alterations and neurodegeneration (Douaud, et al., 2022, Nature 604:697-707), and cerebral microvascular injury (Lee, et al., 2021, Brain 145:2555-2568), in the brains of COVID-19 patients. The findings suggest vascular injury and that the repair process in the brain may set up inflammation.

What drives post-COVID cognitive changes is still a mystery. One hypothesis is that there is persistent immune activation resulting in reduction in cerebral blood flow. Hence, one potential therapy is to restore cerebral blood flow regulation in the brain and/or improve the synchronization of brain functional connectivity networks.

There is a relationship between end-tidal PaCO₂ and oxygen delivery to the brain. An increase from 40 to 50 mmHg in PetCO₂ resulted in an increase of approximately 30% in oxygen delivery (Fortune, et al., J.Trauma 1992; 32: 618-627). There is evidence that increased CO₂ may decrease inflammation, and decreased CO₂ may increase inflammation (Marongiu, et al., Am J Respir Crit Care Med. 2021; 204(8): 933-942; Laffey, et al., Am J Respir Crit Care Med. 2000; 162:2287-2294).

Fisher, et al. in U.S. Pat. No. 6,622,725 described using a rebreathing circuit whereby the step of introducing an alveolar gas induces cerebral vasodilatation, prevents cerebral vasospasm, and provides cerebral protection following subarachnoid hemorrhage, and cerebral trauma. Therefore, increased cerebral blood flow, increased oxygen delivery to the brain, and potential reduction in inflammation from an increased arterial CO₂, may be beneficial in this patient population.

Venous blood returns to the lungs from the muscles and organs with a higher carbon dioxide content than arterial blood, which diffuses into the alveoli. When a person exhales, the first gas that is exhaled comes from the trachea and major bronchi (dead space), which do not participate in gas exchange and therefore have a gas composition similar to the inhaled gas. The gas at the end of the exhalation is considered to have come from the alveoli and reflects the equilibrium CO₂ concentration between the capillaries and the alveoli.

In 2005, a sequential rebreathing method of increasing arterial partial pressure of carbon dioxide (PaCO₂) to a predetermined level using a breathing circuit which allowed for controlled rebreathing was proposed (Somogyi, et al., Anaesth Intensive Care 2005; 33: 726-732). A commercial device, called the Hi-Ox_{SR}, was developed to enable this method. One advantage of the Hi-Ox_{SR} compared to supplying exogenous CO₂ mixtures to breathe is that rebreathing circuits require no source of exogenous gas; the PCO₂ of the reserve gas is self-adjusting and follows the target PetCO₂ levels. Details of the Hi-Ox_{SR} are provided described in the materials and methods. The Hi-Ox was tested by the Defence Research and Development Canada at oxygen flow rates of 0.5 to 4.0 liters per minute (Bouak, et al., DRDC Toronto TM 2006; 201). At all flow rates, the FiO₂ increased, and the mask was determined to be useful for mass casualty situations where oxygen conservation would be beneficial.

Normal end-tidal PCO₂ is in the range of 5-6% (equivalent to approximately 35-45 mmHg). Inhaled CO₂ as a therapeutic treatment has been delivered in concentrations up to 10% CO₂, with the majority of work at 5% CO₂ and without significant adverse effects (Robinson L J, et al., JAMA. 1935;105(22):1734-1738; Priyamvada, et al., J Evolution Med Dent Sci 2018; 7(35):3878-3882).

This sequential rebreathing approach has been used in research and clinical applications to evaluate cerebrovascular reactivity during MRI's and retinal arterial blood flow (Vesely, et al., Magnetic Resonance in Medicine 2001; 45:1011-1013; Mikulis, et al., J Neurosurg. 2005;103(2):347-55; Gilmore, et al., Invest Ophthalmol Vis Sci. 2008; 49:699-705; Gilmore, et al., Am J Physiol Heart Circ Physiol 2005; 288: H2912-H2917). The retinal blood flow research studies included seventy-three (73) subjects and there were no adverse events reported.

Recent publications by El-Betany and Galganska, suggested that inhaled CO₂ could be used as a treatment for patients with SARS-CoV2 infections (El-Bethany, et al., Front Med (Lausanne). 2020; 7: 594295; Galganska, et al., Cell Mol Life Sci 2021; 78(24):8229-8242). El-Betany concluded that, “[d]epending on the therapeutic regime, CO₂ could also ameliorate other COVID-19 symptoms as it has also been reported to have antioxidant, anti-inflammation, anti-cytokine effects, and to stimulate the human immune system.”

Sabel, et al. reported that electrical brain stimulation increased cerebral blood flow and improved oxygen delivery in two subjects with post-COVID cognitive dysfunction (Sabel, et al., Restorative Neurology and Neuroscience. 2021; 39:393-408). After 10 and 13 days respectively, the subjects had significant improvement in cognitive function. They concluded that, “because recovery of function was associated with restoration of vascular autoregulation, we propose that (i) hypometabolic, “silent” neurons are the likely biological cause of long-COVID associated visual and cognitive deficits, and (ii) reoxygenation of these “silent” neurons provides the basis for neural reactivation and neurological recovery.” Moreover, increases in CO₂ level have been found to be associated with increases in serotonin levels (Richerson, 2004, Nat Rev Neuro 5:449-461; Buchanan, et al., 2010, PNAS 107:16354:16359; Corcoran, et al., 2009, Resp Phys & Neuro 168:49-58)).

There is a need in the art for methods of improving cognition, such as for subjects with long COVID. The present invention satisfies this need.

### SUMMARY OF THE INVENTION

In some embodiments, the invention relates to a method of improving cognition in a subject in need thereof comprising inhaling air having an elevated carbon dioxide concentration with a sequential rebreathing device.

In some embodiments, the sequential rebreathing device uses a breathing circuit that stores and then redirects the subject's exhaled gas back to the subject for inhalation following the inhalation of a small volume of oxygen containing an above atmospheric concentration, and a volume which is less than that required to maintain normal ventilation.

In some embodiments, the sequential rebreathing device provides at least about 0.5, 1.0, 1.5, 2.0, 2.5, 3.0, 3.5, 4.0, 4.5, 5.0, 5.5, or 6.0 liters per minute of oxygen.

In some embodiments, the inhaling air having an elevated carbon dioxide concentration results in a PaCO₂ of 35 mmHg, 36 mmHg, 37 mmHg, 38 mmHg, 39 mmHg, 40 mmHg, 41 mmHg, 42 mmHg, 43 mmHg, 44 mmHg, 45 mmHg, 46 mmHg, 47 mmHg, 48 mmHg, 49 mmHg, 50 mmHg, 51 mmHg, 52 mmHg, 53 mmHg, 54 mmHg, or 55 mmHg.

In some embodiments, the subject in need is assessed to exhibit cognitive dysfunction. In some embodiments, the subject in need thereof has, had, is suspected of having, or has been diagnosed with COVID, post-COVID conditions, post-COVID syndrome, long COVID, viral infection, post viral infection, bacterial infection, post bacterial infection, brain inflammation, mild cognitive impairment (MCI), Alzheimer's disease, early onset Alzheimer's disease, dementia, Lewey Body dementia, stroke, transient ischemic attack (TIA), head trauma, concussion, traumatic brain injury (TBI), or age-related cognitive decline. In some embodiments, the inhaling air having an elevated carbon dioxide concentration is performed for at least one period of at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, or at least about 60 minutes. In some embodiments, the inhaling air having an elevated carbon dioxide concentration is performed at least once per day, at least twice per day, at least three times per day, at least four times per day, at least five times per day, or at least six times per day.

In some embodiments, the inhaling air having an elevated carbon dioxide concentration leads to increased blood flow in the brain. In some embodiments, the inhaling air having an elevated carbon dioxide concentration leads to reduced inflammation in the brain. In some embodiments, the inhaling air having an elevated carbon dioxide concentration leads to increased levels of serotonin in the brain.

In some embodiments, cognition is assessed at least one time before treatment. In some embodiments, cognition is assessed at least one time after treatment.

In some embodiments, cognition is assessed using at least one cognitive assessment selected from the group consisting of Self-Administered Gerocognitive Exam (SAGE), Montreal Cognitive Assessment (MoCA), Mini-Mental State Exam (MMSE), Mini-Cog, Memory Impairment Screen (MIS), MIS by telephone (MIS-T), Mental Status Questionnaire (MSQ), 8-item Informant Interview (AD8), Functional Activities Questionnaire (FAQ), 7-Minute Screen (7 MS), Abbreviated Mental Test (AMT), St. Louis University Mental Status Examination (SLUMS), Telephone Instrument for Cognitive Status (TICS), TestMyBrain neurocognitive toolkit, Short Form 36 (SF-36) questionnaire, Brain Fog Questionnaire, and Informant Questionnaire on Cognitive Decline in the Elderly (IQCODE).

In some embodiments, cognition is improved following treatment when the cognitive assessment score determined after treatment is higher than the cognitive assessment score determined before treatment by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, or at least about 150%.

### BRIEF DESCRIPTION OF THE DRAWINGS

The following detailed description of various embodiments of the invention will be better understood when read in conjunction with the appended drawings. For the purpose of illustrating the invention, illustrative embodiments are shown in the drawings. It should be understood, however, that the invention is not limited to the precise arrangements and instrumentalities of the embodiments shown in the drawings.

FIG. 1 depicts a graph depicting oxygen delivery vs PetCO₂.

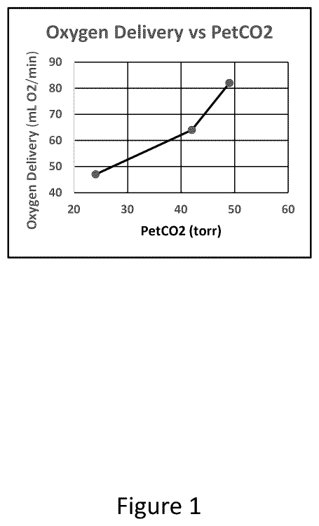

FIG. 2 depicts a patient wearing Hi-Ox_{SR}.

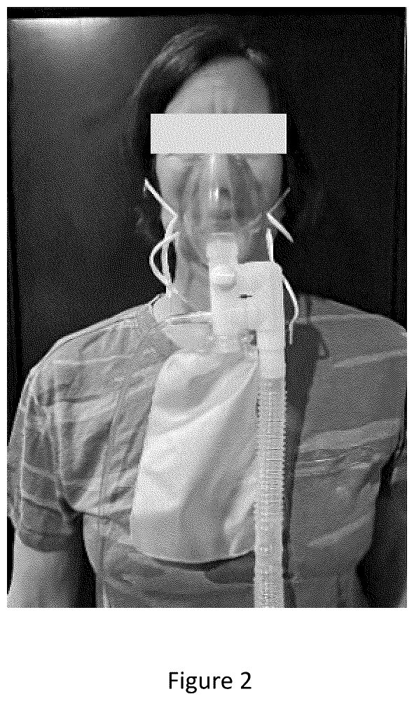

FIG. 3, comprising FIGS. 3A through FIG. 3C, is an illustration of air flow in the Hi-Ox_{SR} oxygen delivery system. In FIG. 3A, the patient exhales their breath into the 1-liter expiratory reservoir tube through the expiratory valve in the valve body. This exhaled gas contains the concentration of the inspired oxygen, less the oxygen uptake per breath or approximately 3-5% less oxygen than was inspired. This reservoir typically contains a high oxygen concentration as well as a 3-5% concentration of carbon dioxide. During this period the oxygen flowing into the valve is directed to the inspiratory reservoir bag and applies pressure to keep the sequential dilution valve closed. In FIG. 3B, when the patient inhales, they breathe in the ˜100% oxygen from the oxygen reservoir bag as well as the low flow oxygen flowing into the valve. In FIG. 3C, once the patient has depleted the oxygen in the reservoir bag, the sequential dilution valve will open, and the patient will inhale the oxygen and carbon dioxide from their previous exhalation. The oxygen flow setting assures a minimum flow of fresh gas to exceed the oxygen uptake requirements of the patient. The intentional reduction in the oxygen flow setting is used to cause targeted increases in partial pressure of carbon dioxide (PaCO₂).

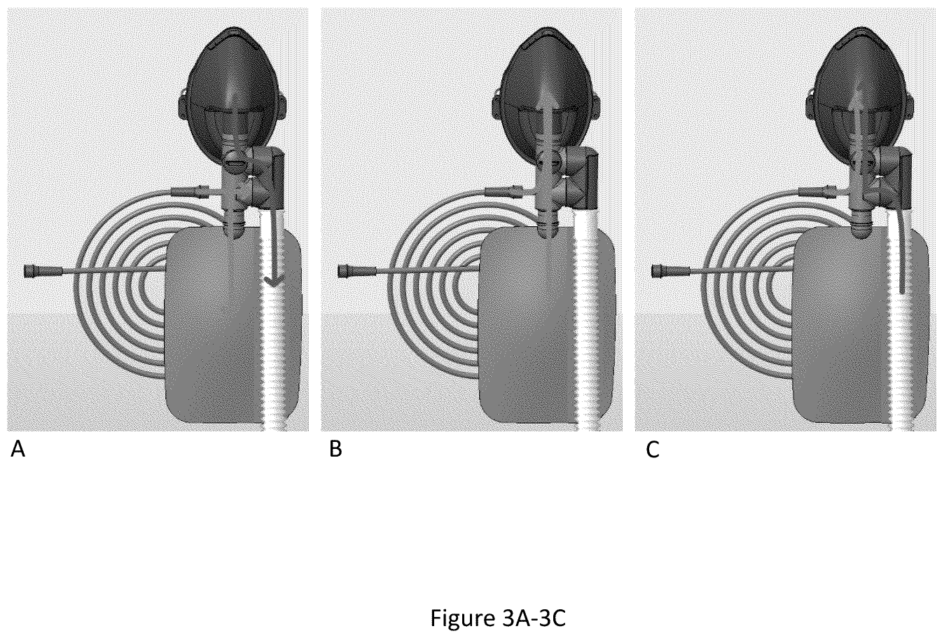

FIG. 4 depicts the results of the cognitive assessments questionnaire. The Day 15 results are the test results from the day following the last treatment. Follow-up was at Day 45.

FIG. 5 depicts the results of the cognitive assessments questionnaire. The Day 15 results are the test results from the day following the last treatment. Follow-up was at Day 45.

FIG. 6 depicts the results of the cognitive assessments questionnaire. The Day 15 results are the test results from the day following the last treatment. Follow-up was at Day 45.

FIG. 7 depicts the results of the cognitive assessments questionnaire. The Day 15 results are the test results from the day following the last treatment. Follow-up was at Day 45.

FIG. 8 depicts the mental component summary (MCS), physical component summary (PCS), and physical functioning (PF) results following treatment.

FIG. 9 depicts the distribution of patients having the indicated MCS, PCS, and PF values at the indicated timepoints following treatment.

FIG. 10 depicts the brief fatigue inventory (BFI) results following treatment.

FIG. 11 depicts the distribution of patients having the indicated BFI at the indicated timepoints following treatment.

### DETAILED DESCRIPTION

The invention is based, at least in part, on the discovery that inhaling air having an elevated carbon dioxide level or concentration leads to improvements in cognition in subjects experiencing cognitive dysfunction or decline associated with a variety of diseases, disorders, and conditions. Thus, in some embodiments, the invention relates to methods of improving cognitive function in a subject in need thereof, comprising inhaling air having an elevated carbon dioxide concentration. In some embodiments, the invention relates to methods of improving cognitive function in a subject in need thereof, comprising inhaling air having an elevated carbon dioxide concentration with a sequential rebreathing device. In some embodiments, the sequential rebreathing device uses a breathing circuit that stores and then redirects the subject's exhaled gas back to the subject for inhalation following the inhalation of a small volume of oxygen containing an above atmospheric concentration, and a volume which is less than that required to maintain normal ventilation.

Advantages of the sequential rebreathing device include but are not limited to no source of exogenous gas being required; a self-adjusting reserve gas PCO₂ which follows the target PetCO₂ level; accessibility; inexpensiveness; and the capability to be self-administered. While no particular sequential rebreathing device is required to practice the methods of the invention described herein, an exemplary sequential rebreathing device can be found described in U.S. Pat. No. 6,622,275.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein and in the appended claims, the singular forms “a,” “or,” and “the’ include plural referents unless the context clearly dictates otherwise.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of at least one sign or symptom of the disease or disorder, the frequency with which at least one sign or symptom is experienced by a patient, or both, is reduced. As used herein, the terms “patient,” “subject,” “individual,” and the like are used interchangeably herein, and refer to any animal, in some embodiments a mammal, and in some embodiments a human, including a human in need of therapy for, or susceptible to, a condition or its sequelae.

A “therapeutic” treatment is a treatment administered to a subject who exhibits at least one sign or symptom of a disease or disorder, for the purpose of diminishing or eliminating at least one sign or symptom of the disease or disorder.

The terms “treat,” “treating,” and “treatment,” refer to therapeutic or preventative measures described herein. The methods of “treatment” employ administration to a subject, in need of such treatment, a method of the present invention, for example, a subject afflicted with a disease or disorder, or a subject who ultimately may acquire such a disease or disorder, in order to prevent, cure, delay, reduce the severity of, reduce the frequency of, or ameliorate at least one sign or symptom of the disease or disorder.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. Reference to “about” a value or parameter herein includes (and describes) variations that are directed to that value or parameter per se. For example, description referring to “about X” includes description of “X”.

It is understood that embodiments described herein include “consisting” and/or “consisting essentially of” embodiments.

### Description

There are several advantages to using a sequential rebreathing device to increase carbon dioxide level or concentration. Non-limiting advantages of the sequential rebreathing device include but are not limited to no source of exogenous gas being required; a self-adjusting reserve gas PCO₂ which follows the target PetCO₂ level; accessibility; inexpensiveness; and the capability to be self-administered.

In some embodiments, the invention is a method of improving cognitive function, improving cognition, reducing cognitive dysfunction, or reducing cognitive decline in a subject in need thereof, comprising inhaling air having an elevated carbon dioxide level or concentration. In some embodiments, the inhaling air having an elevated carbon dioxide level or concentration is performed using a sequential rebreathing device. In some embodiments, the sequential rebreathing device uses a breathing circuit that stores and then redirects the subject's exhaled gas back to the subject for inhalation following the inhalation of a small volume of oxygen containing an above atmospheric concentration, and a volume which is less than that required to maintain normal ventilation.

In some embodiments, the invention is a method of treating a disease or disorder associated with cognitive dysfunction or cognitive decline in a subject in need thereof, comprising inhaling air having an elevated carbon dioxide level or concentration with a sequential rebreathing device. In some embodiments, the sequential rebreathing device uses a breathing circuit that stores and then redirects the subject's exhaled gas back to the subject for inhalation following the inhalation of a small volume of oxygen containing an above atmospheric concentration, and a volume which is less than that required to maintain normal ventilation.

In some embodiments, the invention is a method of treating cognitive dysfunction or reducing cognitive decline in a subject in need thereof, comprising inhaling air having an elevated carbon dioxide level or concentration with a sequential rebreathing device. In some embodiments, the sequential rebreathing device uses a breathing circuit that stores and then redirects the subject's exhaled gas back to the subject for inhalation following the inhalation of a small volume of oxygen containing an above atmospheric concentration, and a volume which is less than that required to maintain normal ventilation.

In some embodiments, the method of inhaling air having an elevated carbon dioxide level or concentration achieves a target partial pressure of carbon dioxide (PaCO₂) of the subject of at least about 35 mmHg, 36 mmHg, 37 mmHg, 38 mmHg, 39 mmHg, 40 mmHg, 41 mmHg, 42 mmHg, 43 mmHg, 44 mmHg, 45 mmHg, 46 mmHg, 47 mmHg, 48 mmHg, 49 mmHg, 50 mmHg, 51 mmHg, 52 mmHg, 53 mmHg, 54 mmHg, or 55 mmHg. In some embodiments, the target partial pressure of carbon dioxide is one or more selected from the group consisting of a target arterial partial pressure and a target end-tidal partial pressure. In some embodiments, the target partial pressure of carbon dioxide is an end-tidal partial pressure.

In various embodiments, the target PaCO₂ of the subject persists for at least about 30 seconds, at least about 1 minute, at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, at least about 11 minutes, at least about 12 minutes, at least about 13 minutes, at least about 14 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, at least about 60 minutes.

In some embodiments, the sequential rebreathing device provides at least about 0.5, 1.0, 1.5, 2.0, 2.5, 3.0, 3.5, 4.0, 4.5, 5.0, 5.5, or 6.0 liters per minute of oxygen.

In some embodiments, the step of inhaling air having an elevated carbon dioxide level or concentration is performed for at least one period lasting at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, or at least about 60 minutes.

In some embodiments, the step of inhaling air having an elevated carbon dioxide level or concentration is performed more than one time. In some embodiments, the step of inhaling air having an elevated carbon dioxide level or concentration is performed more than one time in a day. In various embodiments, the step of inhaling air having an elevated carbon dioxide concentration is performed at least once per day, at least twice per day, at least three times per day, at least four times per day, at least five times per day, or at least six times per day.

In some embodiments, the step of inhaling air having an elevated carbon dioxide level or concentration is performed at least one time on each of more than one day. In some embodiments, the step of inhaling air having an elevated carbon dioxide level or concentration is performed at least one time on more than one consecutive day.

In various embodiments, the step of inhaling air having an elevated carbon dioxide level or concentration is performed for at least 1 day, at least 2 days, at least 3 days, at least 4 days, at least 5 days, at least 6 days, at least 7 days, at least 8 days, at least 9 days, at least 10 days, 11 day, at least 12 days, at least 13 days, at least 14 days, at least 15 days, at least 16 days, at least 17 days, at least 18 days, at least 19 days, at least 20 days, 21 day, at least 22 days, at least 23 days, at least 24 days, at least 25 days, at least 26 days, at least 27 days, at least 28 days, at least 29 days, at least 30 days, at least 31 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 1 year, at least about 2 years, at least about 3 years, at least about 4 years, at least about 5 years, at least about 6 years, at least about 7 years, at least about 8 years, at least about 9 years, or at least about 10 years. In some embodiments, the step of inhaling air having an elevated carbon dioxide concentration is performed at least once per day on non-consecutive days.

In various embodiments, the step of inhaling air having an elevated carbon dioxide concentration is performed at least once per day on at least 2 consecutive days, at least once per day on at least 3 consecutive days, at least once per day on at least 4 consecutive days, at least once per day on at least 5 consecutive days, at least once per day on at least 6 consecutive days, at least once per day on at least 7 consecutive days, at least once per day on at least 8 consecutive days, at least once per day on at least 9 consecutive days, at least once per day on at least 10 consecutive days, at least once per day on at least 11 consecutive days, at least once per day on at least 12 consecutive days, at least once per day on at least 13 consecutive days, at least once per day on at least 14 consecutive days, at least once per day on at least 15 consecutive days, at least once per day on at least 16 consecutive days, at least once per day on at least 17 consecutive days, at least once per day on at least 18 consecutive days, at least once per day on at least 19 consecutive days, at least once per day on at least 20 consecutive days, at least once per day on at least 21 consecutive days, at least once per day on at least 22 consecutive days, at least once per day on at least 23 consecutive days, at least once per day on at least 24 consecutive days, at least once per day on at least 25 consecutive days, at least once per day on at least 26 consecutive days, at least once per day on at least 27 consecutive days, at least once per day on at least 28 consecutive days, at least once per day on at least 29 consecutive days, or at least once per day on at least 30 consecutive days.

It should be understood that after a subject completes a cycle of treatment (by way of non-limiting example, an exemplary cycle of two inhalation sessions per day of 30 minutes per inhalation session for 14 consecutive days) the subject can pause treatment for any number of days (e.g., about 1-360 days or more, or at least about 1, 2, 3, 4, 5, 10, 20, 30, 60, 90, 120, 180 days) and then begin another cycle of treatment.

In various embodiments, the subject has been assessed to be experiencing cognitive dysfunction or cognitive decline. In various embodiments, the subject assessed to be experiencing cognitive dysfunction or cognitive decline has, had, is suspected of having, or has been diagnosed with having at least one disease, disorder, or condition that is associated with cognitive dysfunction or cognitive decline. In some embodiments, the subject assessed to be experiencing cognitive dysfunction or cognitive decline has, had, is suspected of having, or has been diagnosed with at least one of COVID, post-COVID conditions, post-COVID syndrome, long COVID, COVID-related cognitive dysfunction, a viral infection, a past viral infection, a bacterial infection, a past bacterial infection, brain inflammation, mild cognitive impairment (MCI), Alzheimer's disease, early onset Alzheimer's disease, dementia, Lewey Body dementia, stroke, transient ischemic attack (TIA), head trauma, concussion, traumatic brain injury (TBI), age-related cognitive dysfunction or age-related cognitive decline, or a combination thereof.

In some embodiments, cognition, cognitive dysfunction or cognitive decline is assessed using at least one cognitive assessment. Any cognitive assessment can used to assess cognition of subject. By way of non-limiting examples, cognition, cognitive dysfunction or cognitive decline can be assessed using one or more of the Self-Administered Gerocognitive Exam (SAGE), Montreal Cognitive Assessment (MoCA), Mini-Mental State Exam (MMSE), Mini-Cog, Memory Impairment Screen (MIS), MIS by telephone (MIS-T), Mental Status Questionnaire (MSQ), 8-item Informant Interview (AD8), Functional Activities Questionnaire (FAQ), 7-Minute Screen (7 MS), Abbreviated Mental Test (AMT), St. Louis University Mental Status Examination (SLUMS), Telephone Instrument for Cognitive Status (TICS), TestMyBrain neurocognitive toolkit, Short Form 36 (SF-36) questionnaire, Brain Fog Questionnaire, or Informant Questionnaire on Cognitive Decline in the Elderly (IQCODE).

In some embodiments, cognition, cognitive dysfunction or cognitive decline of the subject is assessed at least once before treatment. In some embodiments, cognition, cognitive dysfunction or cognitive decline of the subject is assessed at least once during treatment. In some embodiments, cognition, cognitive dysfunction or cognitive decline of the subject is assessed at least once after treatment.

In some embodiments, cognition is determined to be improved following treatment when the cognitive assessment score determined after treatment is higher than the cognitive assessment score determined before treatment by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, or at least about 150%.

In some embodiments, the methods of improving cognitive function, improving cognition, reducing cognitive dysfunction, by inhaling air having an elevated carbon dioxide concentration leads to increased blood flow in the brain. In some embodiments, the methods of improving cognitive function, improving cognition, reducing cognitive dysfunction, by inhaling air having an elevated carbon dioxide concentration leads to reduced inflammation in the brain. In some embodiments, the methods of improving cognitive function, improving cognition, reducing cognitive dysfunction, by inhaling air having an elevated carbon dioxide concentration leads to increased levels of serotonin in the brain. In some embodiments, the methods of the invention can be used to diminish the dosage of a serotonin-increasing medication (e.g., SSRI, etc.) that is taken by the subject.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out certain embodiments of the present invention, and are not to be construed as limiting in any way the remainder of the disclosure.

### Example 1: Sequential Rebreathing for the Treatment of Long COVID Cognitive Dysfunction

It is known that increases in blood CO₂ will dilate cerebral blood vessels in the brain tissue. Additionally, in the past, while CO₂ was considered simply a waste product of metabolism, recent observations have suggested an important role that CO₂ plays as a signal molecule affecting multiple processes including suppressing inflammatory pathways and inflammatory cytokines.

While not wishing to be bound by any particular theory, the approach described herein takes advantage of the increased cerebral blood flow and anti-inflammatory characteristics of CO₂ to treat subjects with cognitive dysfunction following COVID-19 infections, at least in part by causing an increase in cerebral blood flow to reverse cerebral vascular ischemia and/or inflammation following an infection with SARS-COV-2 by increasing arterial PCO₂. The methods described herein use a breathing circuit which is capable of organizing exhaled gas so as to be preferentially inhaled during rebreathing when the oxygen in the inspired reservoir bag is depleted on each breath. Such a previously described circuit (see U.S. Pat. No. 6,622,725, titled “Rebreathing circuit to set and stabilize end tidal and arterial PCO₂ despite varying levels of minute ventilation”) is set up to use about 1 to 1.5 liters per minute of oxygen, with the balance of the gas for ventilation coming from the rebreathing tube on the exhalation port.

In these studies, the rebreathing tube volume was reduced from 3 liters to about 1 liter. This reduction in oxygen flow, well below the normal flow of oxygen into a mask, is still a safe flow. The average adult human absorbs approximately 0.3 liters of oxygen per minute. By providing about 1 to 1.5 liters per minute (LPM) of oxygen, and with the oxygen entering the alveoli before the rebreathed air, it assures more than sufficient oxygen to sustain the oxygen needs of people being treated.

The Hi-Ox_{SR} High FiO₂ Mask with use of its Sequential Rebreathing Reservoir is designed to create a partial rebreathing oxygen mask that has more control of the sequence of rebreathing exhaled gas and limits the dilution of inspired oxygen with room air.

The Hi-Ox_{SR} has an expiratory valve into a valve body that is attached to the mask and with the addition of a separate expiratory gas rebreathing reservoir there is no contamination of the inspired oxygen. The mask has no holes to dilute the inspiratory gas. When the patient begins inspiration, all their inspired gas is ˜100% oxygen from the inspiratory reservoir. Only when there is no more oxygen available to them from the inspired reservoir, does the expiratory reservoir sequentially contribute to the inspired gas. This late contribution gas contains the high oxygen and a high concentration of carbon dioxide that was exhaled from the previous breath. This gas will return the previously exhaled CO₂ to the lung and limit the reduction in alveolar CO₂.

### Study 1

Five patients expressing symptoms of post-COVID cognitive dysfunction (“brain fog”) were given the Self-Administered Gerocognitive Exam (SAGE) cognitive function test (Scharre, et al., Wexner Medical Center, The Ohio State University). This is a validated and accepted screening test for cognitive dysfunction. Three physicians, licensed to practice medicine in the US, prescribed treatment with the Hi-Ox_{SR} using low flow oxygen (1.5 to 2.5 LPM) for these patients.

The patients had the Hi-Ox_{SR}, oxygen concentrator and pulse oximeter at home for self-treatment. Subjects administered twice-daily treatments of 30 minutes each for 14 days. Oxygen was delivered from the oxygen concentrator at 1.5 LPM to the Hi-Ox_{SR}. If the patient expressed an inability to tolerate that low a flow, they were instructed to increase the oxygen flow in 0.5 LPM increments up to 2.5 LPM.

Patients continuously monitored themselves with a pulse oximeter during treatment. The pulse oximeter served as a safety measure for a tubing disconnection from the concentrator as well as provided assurances to the patients that they were receiving adequate oxygen in the face of a sense of shortness of breath resulting from the rebreathing.

Following completion of the 14-day treatment series, patients were given the SAGE test again to evaluate their post treatment cognitive function. Patients were also asked for their self-perception of changes in brain fog.

Table 1 shows the initial SAGE scores and the post treatment scores following 14 days of treatment with the Hi-Ox_{SR} for 30 minutes twice a day.

TABLE 1

| | | | Pre-Treatment | Post-Treatment | |
|---|---|---|---|---|---|
| Patient | Age | Gender | SAGE | SAGE | Comments |
| 1 | 65 | F | 15 | 22 | Pre MRI revealed mild |
| | | | | | cerebral ischemia. Clear |
| | | | | | subjective improvement. |
| 2 | 69 | F | 17 | 21 | Clear subjective |
| | | | | | improvement. |
| 3 | 71 | M | 11 | 15 | Hx of Alzheimer's. Some |
| | | | | | subjective improvement. |
| 4 | 65 | F | 11 | 18 | Clear subjective |
| | | | | | improvement. |
| 5 | 57 | M | 22 | 22 | Loss of taste returned on |
| | | | | | Day 5. Clear subjective |
| | | | | | improvement in ability to |
| | | | | | recall names, etc. |

Note:
SAGE Scores less than 17 are abnormal. Maximum score is 22.

Most patients reported mild to moderate discomfort rebreathing CO₂, however no subject stopped treatment due to this discomfort. One patient reported a slight increase in blood pressure at a flow of 1.5 LPM, which disappeared at 2.5 LPM. There were no other adverse events in any patient. All patients reported increased oxygen saturation (98-100%) during the treatment as measured with the finger pulse oximeter. All patients provided written consent for their data to be collected and published.

### Study 2

A notable advantage of the Hi-Ox_{SR} rebreathing treatment is its economical aspect, as Hi-Ox_{SR} rebreathers are accessible and inexpensive. Furthermore, the therapy can be self-administered by patients at home.

A single-arm pilot project was conducted to evaluate the safety of re-breathing CO₂ (using the Hi-Ox_{SR} device) for the treatment of cognitive dysfunction in post-COVID conditions (PCC) in an open label study.

Subjects administered twice-daily treatments of 30 minutes each for 14 days.

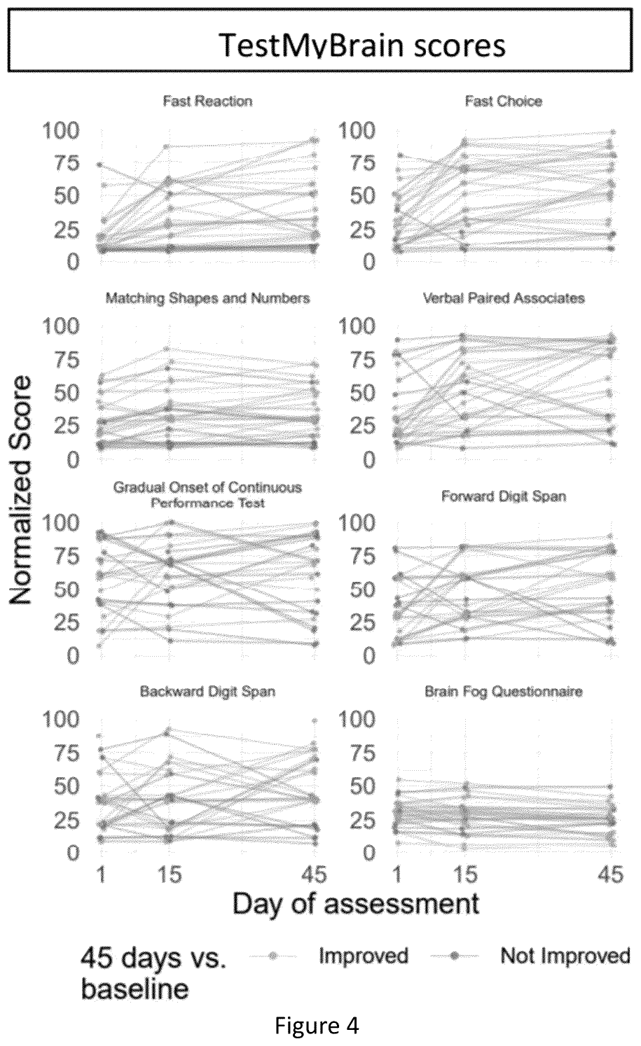

Cognitive assessments questionnaires were completed at baseline, day 15 and day 45. Significant improvements were found between baseline and days 15 and 45 in multiple TestMyBrain tasks (FIG. 4), the Brain Fog Questionnaire.

Of note, some participants in the pilot study were interested in continuing for longer duration. Since the pilot study was small, unblinded and lacked a control group, the positive findings must be confirmed. This pilot study did not provide information on treatment duration exceeding 2 weeks, so it is unclear if a longer duration of Hi-Ox_{SR} treatment would yield larger improvements.

As such, this trial expanded the pilot study findings to implement a phase 2 trial assessing the use of Hi-Ox_{SR} as an intervention for cognitive dysfunction in PCC. This was the first RCT assessing this novel use of Hi-Ox_{SR} for the treatment of cognitive dysfunction in PCC.

Given the above findings along with the excellent safety profile of Hi-Ox_{SR}, this trial has high potential to support the development and commercialization of a new indication for Hi-Ox_{SR} and to respond to an urgent global health challenge.

### Example 2: Pilot Open Label Use of the Hi-Ox_{SR} for the Treatment of Post COVID Cognitive Dysfunction

Approximately 45% of patients with post-COVID condition (PCC) report brain fog. One proposed mechanism is persistent immune activation following SARS-COV-2 infection, resulting in reduction in cerebral blood flow and brain damage. CO₂ has been proposed as a potential treatment due to its antioxidant, anti-inflammatory, and vasodilatory effects. As such, a pilot study was conducted to assess the open label use of re-breathing CO₂ using Hi-Ox_{SR} mask for the treatment of post-COVID cognitive dysfunction.

A pilot/phase 1 study was performed to assess the safety and efficacy of Hi-Ox_{SR} treatment for post-COVID cognitive dysfunction.

Adult participants (≥18) with post-COVID cognitive dysfunction were enrolled in this study. Participants were instructed to use are breathing mask at low oxygen flow (0.5 to 2.0 L per min) (Hi-Ox_{SR}) twice a day for 30 minutes for 14consecutive days. They were asked to report any adverse effects and complete neurocognitive tests, including TestMyBrain neurocognitive toolkit, SF-36, and Brain Fog Questionnaire at baseline, day 15 and 45.

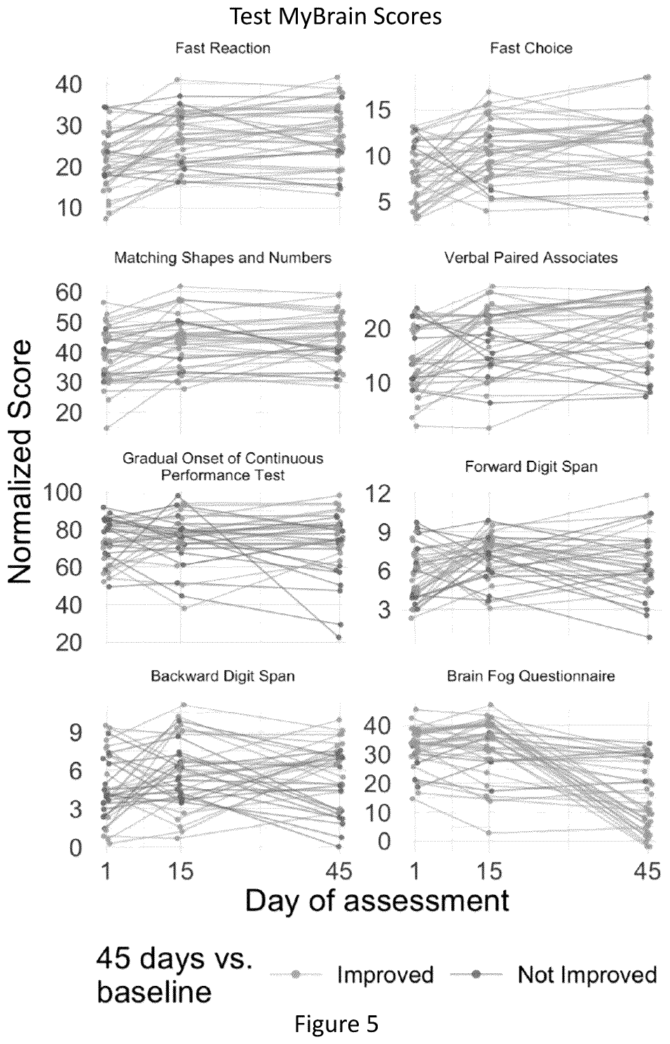

A total of 36 participants completed the study. There were no severe adverse effects. Common adverse effects include shortness of breath, nausea, and headaches. Significant improvements were found between baseline and Days 15 and 45 in multiple TestMyBrain categories (FIG. 5), the Brain Fog Questionnaire, and SF-36 mental and physical composite scores.

The pilot study showed Hi-Ox_{SR} to be a safe and effective treatment for post-COVID cognitive dysfunction. Larger controlled trials are needed to assess the clinical utility of this treatment for post-COVID cognitive dysfunction.

### Example 3: HiOx Phase II

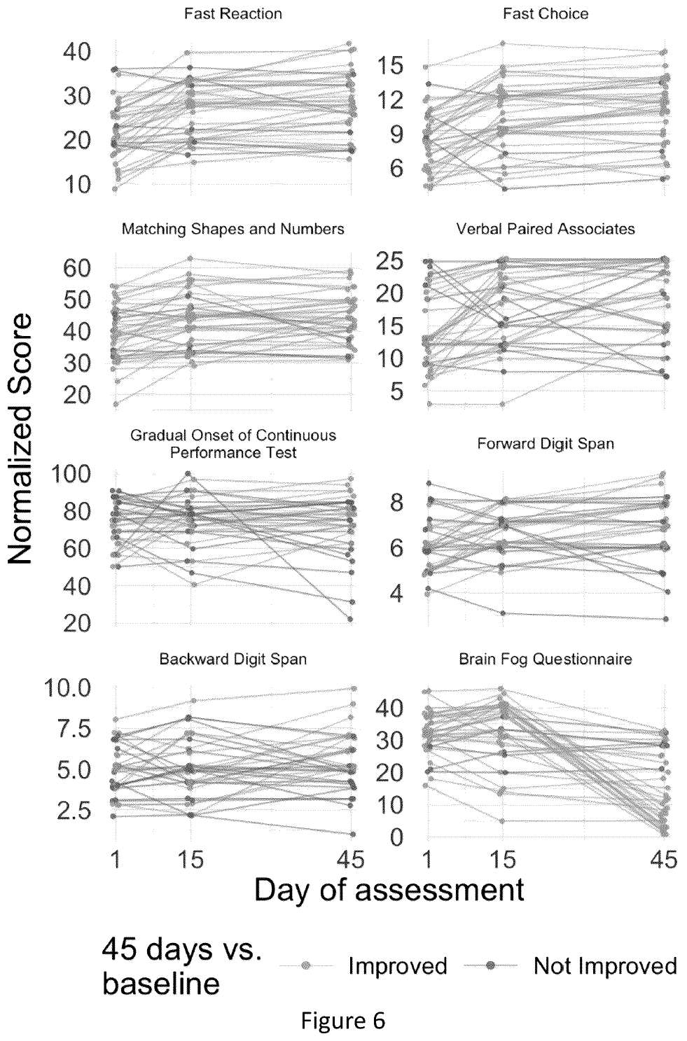

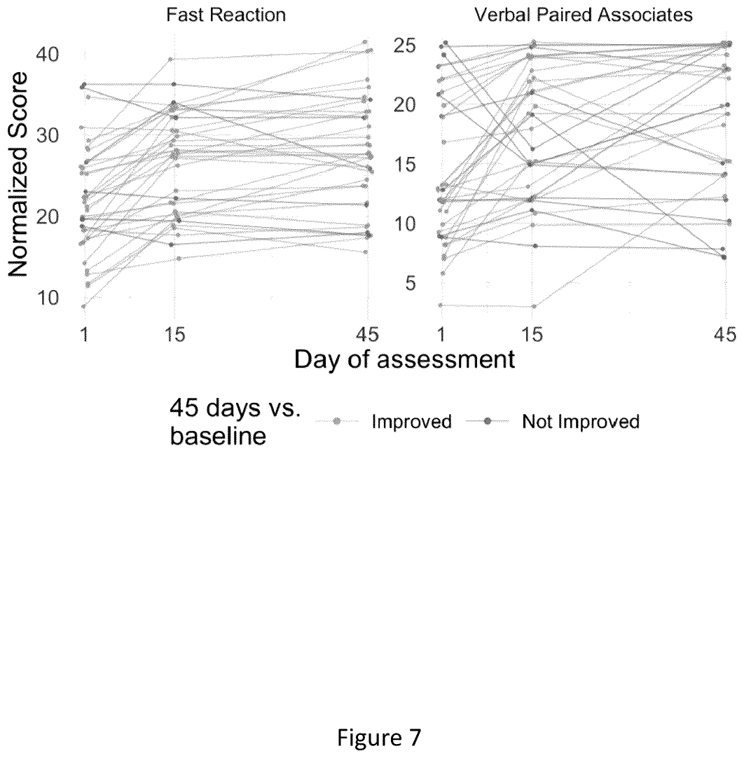

Scored TMB data (and MSNQ)—individual trajectories over time on the pilot study—are described herein. All 8 outcomes are depicted (FIGS. 6 and 7). The comparisons are of Day 15 (“15”) and Day 45 (“45”) to baseline (“BL”). The following Tables describe simple proportions with better, worse, and same scores for the indicated outcome variables.

TABLE 2
Improvement or worsening over time in Fast Reaction.

| | Worsened | Unchanged | Improved | Unknown | Total |
|---|---|---|---|---|---|
| Change BL to 15 | 7 (18.9%) | 0 (0.0%) | 29 (78.4%) | 1 (2.7%) | 37 |
| Change BL to 45 | 6 (16.2%) | 0 (0.0%) | 30 (81.1%) | 1 (2.7%) | 37 |
| Change 15 to 45 | 14 (37.8%) | 0 (0.0%) | 22 (59.5%) | 1 (2.7%) | 37 |

TABLE 3
Improvement or worsening over time in Fast Choice.

| | Worsened | Unchanged | Improved | Unknown | Total |
|---|---|---|---|---|---|
| Change BL to 15 | 4 (10.8%) | 0 (0.0%) | 32 (86.5%) | 1 (2.7%) | 37 |
| Change BL to 45 | 3 (8.1%) | 0 (0.0%) | 33 (89.2%) | 1 (2.7%) | 37 |
| Change 15 to 45 | 11 (29.7%) | 0 (0.0%) | 25 (67.6%) | 1 (2.7%) | 37 |

TABLE 4
Improvement or worsening over time in Matching Shapes and Numbers.

| | Worsened | Unchanged | Improved | Unknown | Total |
|---|---|---|---|---|---|
| Change BL to 15 | 7 (18.9%) | 1 (2.7%) | 28 (75.7%) | 1 (2.7%) | 37 |
| Change BL to 45 | 4 (10.8%) | 1 (2.7%) | 31 (83.8%) | 1 (2.7%) | 37 |
| Change 15 to 45 | 15 (42.9%) | 0 (0.0%) | 19 (54.3%) | 1 (2.9%) | 35 |

TABLE 5
Improvement or worsening over time in Verbal Paired Associates.

| | Worsened | Unchanged | Improved | Unknown | Total |
|---|---|---|---|---|---|
| Change BL to 15 | 5 (13.5%) | 4 (10.8%) | 27 (73.0%) | 1 (2.7%) | 37 |
| Change BL to 45 | 7 (18.9%) | 4 (10.8%) | 25 (67.6%) | 1 (2.7%) | 37 |
| Change 15 to 45 | 10 (32.3%) | 2 (6.5%) | 18 (58.1%) | 1 (3.2%) | 31 |

TABLE 6
Improvement or worsening over time in Gradual
Onset of Continuous Performance Test.

| | Worsened | Unchanged | Improved | Unknown | Total |
|---|---|---|---|---|---|
| Change BL to 15 | 13 (35.1%) | 6 (16.2%) | 16 (43.2%) | 2 (5.4%) | 37 |
| Change BL to 45 | 13 (35.1%) | 4 (10.8%) | 18 (48.6%) | 2 (5.4%) | 37 |
| Change 15 to 45 | 14 (42.4%) | 2 (6.1%) | 16 (48.5%) | 1 (3.0%) | 33 |

TABLE 7
Improvement or worsening over time in Forward Digit Span.

| | Worsened | Unchanged | Improved | Unknown | Total |
|---|---|---|---|---|---|
| Change BL to 15 | 7 (18.9%) | 12 (32.4%) | 16 (43.2%) | 2 (5.4%) | 37 |
| Change BL to 45 | 6 (16.2%) | 6 (16.2%) | 23 (62.2%) | 2 (5.4%) | 37 |
| Change 15 to 45 | 4 (16.0%) | 3 (12.0%) | 16 (64.0%) | 2 (8.0%) | 25 |

TABLE 8
Improvement or worsening over time in Backward Digit Span.

| | Worsened | Unchanged | Improved | Unknown | Total |
|---|---|---|---|---|---|
| Change BL to 15 | 9 (24.3%) | 13 (35.1%) | 13 (35.1%) | 2 (5.4%) | 37 |
| Change BL to 45 | 6 (16.2%) | 10 (27.0%) | 19 (51.4%) | 2 (5.4%) | 37 |
| Change 15 to 45 | 13 (39.4%) | 4 (12.1%) | 15 (45.5%) | 1 (3.0%) | 33 |

TABLE 9
Improvement or worsening over time in Brain Fog Questionnaire.

| | Worsened | Unchanged | Improved | Unknown | Total |
|---|---|---|---|---|---|
| Change BL to 15 | 21 (56.8%) | 4 (10.8%) | 11 (29.7%) | 1 (2.7%) | 37 |
| Change BL to 45 | 2 (5.4%) | 3 (8.1%) | 31 (83.8%) | 1 (2.7%) | 37 |
| Change 15 to 45 | 4 (11.1%) | 0 (0.0%) | 31 (86.1%) | 1 (2.8%) | 36 |

TABLE 10
Summaries at each time.

| Variable | Time 1 | Time 15 | Time 45 |
|---|---|---|---|
| Fast Reaction | 21.8 (6.8) | 26.6 (6.4) | 27.8 (7.1) |
| | [21.0; 17.4-26.3]; n = 37 | [27.6; 20.3-32.2]; n = 36 | [27.7; 23.1-32.7]; n = 36 |
| Fast Choice | 8.3 (2.7) | 10.3 (3.1) | 10.9 (2.9) |
| | [8.2; 6.0-10.3]; n = 37 | [10.0; 8.8-12.4]; n = 36 | [11.3; 8.7-13.1]; n = 36 |
| Matching Shapes and | 38.9 (8.8) | 43.2 (8.6) | 43.7 (7.8) |
| Numbers | [39; 32.0-46.0]; n = 37 | [44; 35.8-47.2]; n-36 | [44; 37.8-49.0]; n = 36 |
| Verbal Paired | 14.1(6.1) | 17.6 (5.9) | 19.0 (6.1) |
| Associates | [12.0; 9.0-20.0]; n = 37 | [18.5; 12.0-23.2]; n = 36 | [21.0; 14.0-25.0]; n = 36 |
| Gradual Onset of | 73.2 (11.7) | 75.1 (12.9) | 72.7 (16.3) |
| Continuous | [75.0; 65.6-84.4]; n-36 | [78.1; 71.1-81.2]; n = 36 | [76.6; 67.0-84.4]; n = 36 |
| Performance Test | | | |
| Forward Digit Span | 6.1 (1.2) | 6.5 (1.1) | 6.8 (1.4) |
| | [6; 5.0-7]; n = 36 | [7; 6.0-7]; n = 36 | [7; 6.0-8]; n = 36 |
| Backward Digit Span | 4.8 (1.5) | 5.0 (1.8) | 5.4 (1.8) |
| | [5; 4.0-6.0]; n = 36 | [5; 4.0-6.0]; n = 36 | [5; 4.0-6.2]; n-36 |
| Brain Fog | 31.7 (6.7) | 32.3 (9.8) | 14.8 (11.3) |
| Questionnaire | [32.0; 28.0-37.0]; n = 37 | [33.0; 27.5-40.0]; n = 36 | [11.5; 4.5-25.8]; n = 36 |

Summary statistics (mean (SD) [Median; IQR], n) for each outcome at each time.

The following Tables describe changes in mean scores using linear mixed models for the indicated outcome variables. Random effects for individuals are shown. Categorical variables for Day 15 (“15”) and Day 45 (“45”) (Baseline (“BL”) as the reference) are shown. “Estimate” is denoted as “est.”

TABLE 11
Outcome variable: Fast Reaction.

| | est. | lower | upper | p. value |
|---|---|---|---|---|
| BL | 21.8 | 19.6 | 24.0 | <0.0001 |
| Change to 15 | 4.7 | 3.1 | 6.3 | <0.0001 |
| Change to 45 | 5.9 | 4.3 | 7.5 | <0.0001 |

Between-person SD at baseline is 6.8, so the mean changes are 70% & 88% of an SD.

TABLE 12
Outcome variable: Fast Reaction.

| | est. | lower | upper | p. value |
|---|---|---|---|---|
| Change to 15 | 70 | 46 | 93 | <0.0001 |
| Change to 45 | 88 | 64 | 111 | <0.0001 |

Changes as percentages of baseline SD of 6.8.

TABLE 13
Outcome variable: Fast Reaction.

| Comparison | Correlation |
|---|---|
| BL and 15 | 0.75 |
| BL and 45 | 0.65 |
| 15 and 45 | 0.87 |

Correlations for Fast Reaction.

TABLE 14
Outcome variable: Fast Reaction.

| | est. | lower | upper | p. value |
|---|---|---|---|---|
| BL | 8.3 | 7.4 | 9.3 | <0.0001 |
| Change to 15 | 1.9 | 1.3 | 2.6 | <0.0001 |
| Change to 45 | 2.4 | 1.8 | 3.1 | <0.0001 |

Between-person SD at baseline is 2.7, so the mean changes are 71% & 91% of an SD.

TABLE 15
Outcome variable: Fast Reaction.

| | est. | lower | upper | p. value |
|---|---|---|---|---|
| Change to 15 | 71 | 47 | 95 | <0.0001 |
| Change to 45 | 91 | 67 | 115 | <0.0001 |

Changes as percentages of baseline SD of 2.7.

TABLE 16
Outcome variable: Fast Reaction.

| Comparison | Correlation |
|---|---|
| BL and 15 | 0.71 |
| BL and 45 | 0.68 |
| 15 and 45 | 0.92 |

Correlations for Fast Choice.

TABLE 17
Outcome variable: Matching Shapes and Numbers.

| | est. | lower | upper | p. value |
|---|---|---|---|---|
| BL | 38.9 | 36.2 | 41.7 | <0.0001 |
| Change to 15 | 4.0 | 2.2 | 5.9 | 0.0001 |
| Change to 45 | 4.6 | 2.7 | 6.4 | <0.0001 |

Between-person SD at baseline is 8.8, so the mean changes are 46% & 52% of an SD.

TABLE 18
Outcome variable: Matching Shapes and Numbers.

| | est. | lower | upper | p. value |
|---|---|---|---|---|
| Change to 15 | 46 | 25 | 67 | 0.0001 |
| Change to 45 | 52 | 31 | 73 | <0.0001 |

Changes as percentages of baseline SD of 8.8.

TABLE 19
Outcome variable: Matching Shapes and Numbers.

| Comparison | Correlation |
|---|---|
| BL and 15 | 0.82 |
| BL and 45 | 0.82 |
| 15 and 45 | 0.70 |

Correlations for Matching Shapes and Numbers.

TABLE 20
Outcome variable: Verbal Paired Associates.

| | est. | lower | upper | p. value |
|---|---|---|---|---|
| BL | 14.1 | 12.2 | 16.1 | <0.0001 |
| Change to 15 | 3.4 | 1.5 | 5.2 | 0.0005 |
| Change to 45 | 4.8 | 3.0 | 6.7 | <0.0001 |

Between-person SD at baseline is 6.1, so the mean changes are 55% & 79% of an SD.

TABLE 21
Outcome variable: Verbal Paired Associates.

| | est. | lower | upper | p. value |
|---|---|---|---|---|
| Change to 15 | 55 | 25 | 85 | 0.0005 |
| Change to 45 | 79 | 49 | 109 | <0.0001 |

Changes as percentages of baseline SD of 6.1.

TABLE 22
Outcome variable: Verbal Paired Associates.

| Comparison | Correlation |
|---|---|
| BL and 15 | 0.61 |
| BL and 45 | 0.49 |
| 15 and 45 | 0.62 |

Correlations for Verbal Paired Associates.

TABLE 23
Outcome variable: Gradual Onset of Continuous Performance Test.

| | est. | lower | upper | p. value |
|---|---|---|---|---|
| BL | 73.0 | 68.5 | 77.6 | <0.0001 |
| Change to 15 | 2.0 | − 3.1 | 7.1 | 0.4357 |
| Change to 45 | − 0.4 | − 5.5 | 4.7 | 0.8732 |

Between-person SD at baseline is 11.7, so the mean changes are 17% & 3% of an SD.

TABLE 24
Outcome variable: Gradual Onset of Continuous Performance Test.

| | est. | lower | upper | p. value |
|---|---|---|---|---|
| Change to 15 | 17 | −26 | 60 | 0.4357 |
| Change to 45 | −3 | −47 | 40 | 0.8732 |

Changes as percentages of baseline SD of 11.7.

TABLE 25
Outcome variable: Gradual Onset of Continuous Performance Test.

| Comparison | Correlation |
|---|---|
| BL and 15 | 0.42 |
| BL and 45 | 0.49 |
| 15 and 45 | 0.33 |

Correlations for Gradual Onset of Continuous Performance Test.

TABLE 26
Outcome variable: Forward Digit Span.

| | est. | lower | upper | p. value |
|---|---|---|---|---|
| BL | 6.1 | 5.7 | 6.5 | <0.0001 |
| Change to 15 | 0.4 | 0.1 | 0.8 | 0.0230 |
| Change to 45 | 0.7 | 0.4 | 1.1 | 0.0001 |

Between-person SD at baseline is 1.2, so the mean changes are 35% & 61% of an SD.

TABLE 27
Outcome variable: Forward Digit Span.

| | est. | lower | upper | p. value |
|---|---|---|---|---|
| Change to 15 | 35 | 5 | 66 | 0.0230 |
| Change to 45 | 61 | 31 | 92 | 0.0001 |

Changes as percentages of baseline SD of 1.2.

TABLE 28
Outcome variable: Forward Digit Span.

| Comparison | Correlation |
|---|---|
| BL and 15 | 0.64 |
| BL and 45 | 0.54 |
| 15 and 45 | 0.71 |

Correlations for Forward Digit Span.

TABLE 29
Outcome variable: Backward Digit Span.

| | est. | lower | upper | p. value |
|---|---|---|---|---|
| BL | 4.8 | 4.2 | 5.3 | <0.0001 |
| Change to 15 | 0.2 | −0.3 | 0.7 | 0.4207 |
| Change to 45 | 0.5 | 0.0 | 1.0 | 0.0379 |

Between-person SD at baseline is 1.5, so the mean changes are 13% & 35% of an
SD.

TABLE 30
Outcome variable: Backward Digit Span.

| | est. | lower | upper | p. value |
|---|---|---|---|---|
| Change to 15 | 13 | −20 | 47 | 0.4207 |
| Change to 45 | 35 | 2 | 68 | 0.0379 |

Changes as percentages of baseline SD of 1.5.

TABLE 31
Outcome variable: Backward Digit Span.

| Comparison | Correlation |
|---|---|
| BL and 15 | 0.69 |
| BL and 45 | 0.70 |
| 15 and 45 | 0.47 |

Correlations for Backward Digit Span.

TABLE 32
Outcome variable: Brain Fog Questionnaire.

| | est. | lower | upper | p. value |
|---|---|---|---|---|
| BL | 31.7 | 28.6 | 34.8 | 0.0001 |
| Change to 15 | 0.5 | −3.5 | 4.6 | 0.7949 |
| Change to 45 | −17.0 | −21.1 | −12.9 | <0.0001 |

Between-person SD at baseline is 6.7, so the mean changes are 8% & 255% of an SD.

TABLE 33
Outcome variable: Brain Fog Questionnaire.

| | est. | lower | upper | p. value |
|---|---|---|---|---|
| Change to 15 | 8 | 53 | 69 | 0.7949 |
| Change to 45 | −255 | −316 | −194 | <0.0001 |

Changes as percentages of baseline SD of 6.7.

TABLE 34
Outcome variable: Brain Fog Questionnaire.

| Comparison | Correlation |
|---|---|
| BL and 15 | 0.76 |
| BL and 45 | 0.03 |
| 15 and 45 | −0.12 |

Correlations for Brain Fog Questionnaire.

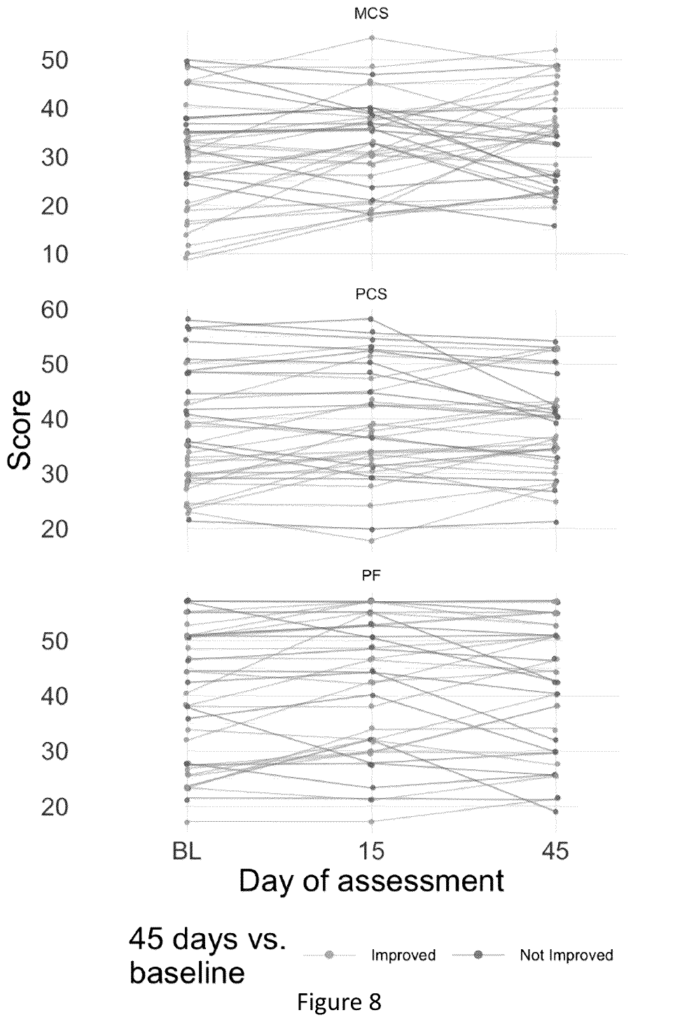

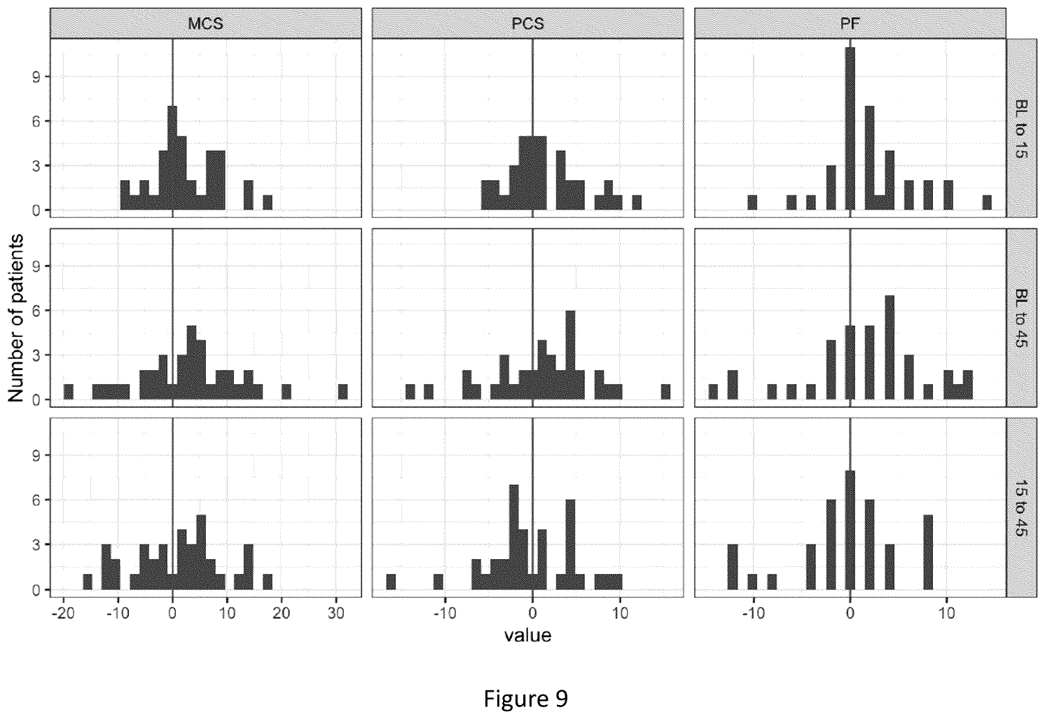

FIGS. 8 and 9 and following tables describe results from SF-36.

TABLE 35
Summary statistics for change in SF-36.

| | | | | | | |
|---|---|---|---|---|---|---|
| n | Change | Mean | SD | CI. low | CI. high | p. value |

MCS

| | | | | | | |
|---|---|---|---|---|---|---|
| NA | BL to 15 | 2.3 | 6.2 | 0.2 | 4.4 | 0.0353 |
| NA | BL to 45 | 3.1 | 9.9 | −0.3 | 6.5 | 0.0687 |
| NA | 15 to 45 | 0.9 | 8.4 | −2.0 | 3.7 | 0.5469 |

PCS

| | | | | | | |
|---|---|---|---|---|---|---|
| NA | BL to 15 | 1.4 | 4.2 | 0.0 | 2.9 | 0.0486 |
| NA | BL to 45 | 1.0 | 5.9 | −1.0 | 3.1 | 0.3006 |
| NA | 15 to 45 | −0.4 | 5.2 | −2.2 | 1.4 | 0.6572 |

PF

| | | | | | | |
|---|---|---|---|---|---|---|
| NA | BL to 15 | 2.0 | 4.8 | 0.4 | 3.6 | 0.0166 |
| NA | BL to 45 | 1.6 | 6.8 | −0.7 | 3.9 | 0.1631 |
| NA | 15 to 45 | −0.4 | 5.8 | −2.4 | 1.5 | 0.6734 |

TABLE 36
Summary statistics for categorized change in SF-36.

| | | |
|---|---|---|
| 1. Decrease > | 2. Change −5 | 3. Increase > |
| 5 points | to 5 | 5 points |

MCS

| | | | |
|---|---|---|---|
| BL to 15 | 5 (13.9%) | 20 (55.6%) | 11 (30.6%) |
| BL to 45 | 6 (16.7%) | 17 (47.2%) | 13 (36.1%) |
| 15 to 45 | 10 (27.8%) | 14 (38.9%) | 12 (33.3%) |

PCS

| | | | |
|---|---|---|---|
| BL to 15 | 1 (2.8%) | 28 (77.8%) | 7 (19.4%) |
| BL to 45 | 5 (13.9%) | 25 (69.4%) | 6 (16.7%) |
| 15 to 45 | 4 (11.1%) | 28 (77.8%) | 4 (11.1%) |

PF

| | | | |
|---|---|---|---|
| BL to 15 | 2 (5.6%) | 27 (75.0%) | 7 (19.4%) |
| BL to 45 | 5 (13.9%) | 22 (61.1%) | 9 (25.0%) |
| 15 to 45 | 5 (13.9%) | 26 (72.2%) | 5 (13.9%) |

The following Tables describe changes in mean scores using linear mixed models for the indicated outcome variables.

TABLE 37
Outcome variable: PCS.

| | est. | lower | upper | p. value |
|---|---|---|---|---|
| BL | 37.5 | 34.2 | 40.7 | <0.0001 |
| Change to 15 | 1.5 | −0.3 | 3.2 | 0.0961 |
| Change to 45 | 1.1 | −0.7 | 2.8 | 0.2211 |

Between-person SD at baseline is 10.5, so the mean changes are 14% & 10% of an SD.

TABLE 38
Outcome variable: PCS.

| | est. | lower | upper | p. value |
|---|---|---|---|---|
| Change to 15 | 14 | −3 | 30 | 0.0961 |
| Change to 45 | 10 | −6 | 26 | 0.2211 |

Changes as percentages of baseline SD of 10.5.

TABLE 39
Outcome variable: PCS.

| Comparison | Correlation |
|---|---|
| BL and 15 | 0.92 |
| BL and 45 | 0.83 |
| 15 and 45 | 0.86 |

Correlation for PCS.

TABLE 40
Outcome variable: MCS.

| | est. | lower | upper | p. value |
|---|---|---|---|---|
| BL | 30.3 | 26.9 | 33.6 | <0.0001 |
| Change to 15 | 2.3 | −0.5 | 5.0 | 0.1072 |
| Change to 45 | 3.1 | 0.4 | 5.9 | 0.0276 |

Between-person SD at baseline is 11.1, so the mean changes are 20% & 28% of an SD.

TABLE 41
Outcome variable: MCS.

| | est. | lower | upper | p. value |
|---|---|---|---|---|
| Change to 15 | 20 | −5 | 45 | 0.1072 |
| Change to 45 | 28 | 3 | 53 | 0.0276 |

Changes as percentages of baseline SD of 11.1.

TABLE 42
Outcome variable: MCS.

| Comparison | Correlation |
|---|---|
| BL and 15 | 0.83 |
| BL and 45 | 0.56 |
| 15 and 45 | 0.62 |

Correlations for MCS.

TABLE 43
Outcome variable: PF.

| | est. | lower | upper | p. value |
|---|---|---|---|---|
| BL | 39.7 | 35.7 | 43.6 | <0.0001 |
| Change to 15 | 2.0 | 0.1 | 4.0 | 0.0394 |
| Change to 45 | 1.6 | −0.3 | 3.6 | 0.0978 |

Between-person SD at baseline is 12.2, so the mean changes are 17% & 13% of an SD.

TABLE 44
Outcome variable: PF.

| | est. | lower | upper | p. value |
|---|---|---|---|---|
| Change to 15 | 17 | 1 | 33 | 0.0394 |
| Change to 45 | 13 | −3 | 29 | 0.0978 |

Changes as percentages of baseline SD of 12.2.

TABLE 45
Outcome variable: PF.

| Comparison | Correlation |
|---|---|
| BL and 15 | 0.92 |
| BL and 45 | 0.85 |
| 15 and 45 | 0.89 |

Correlations for PF.

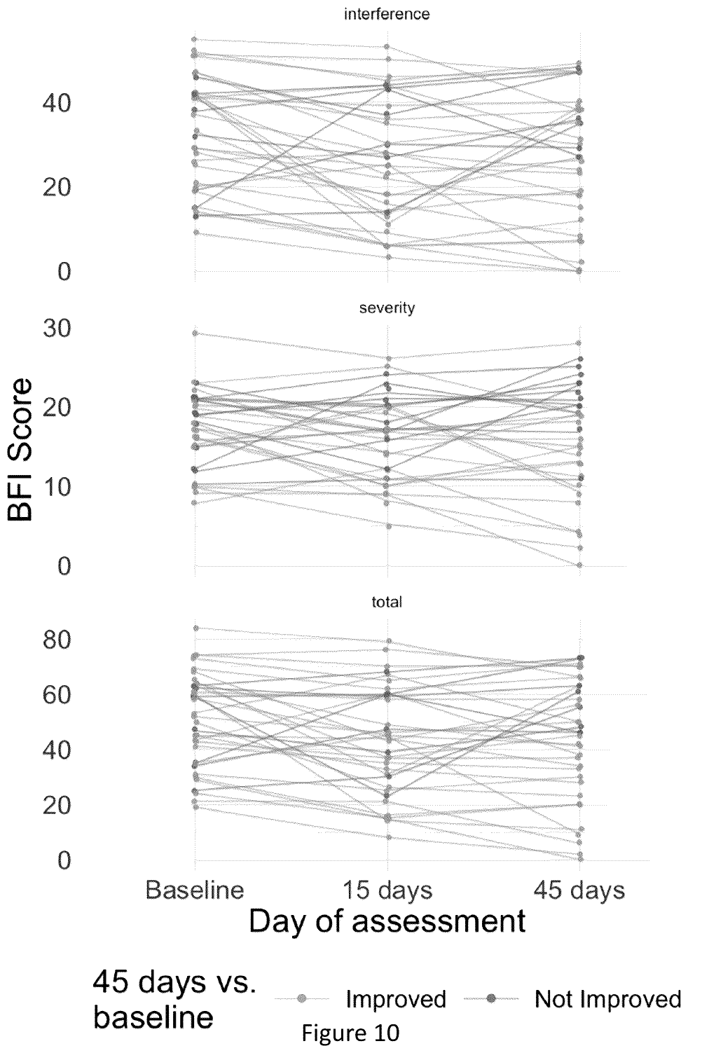

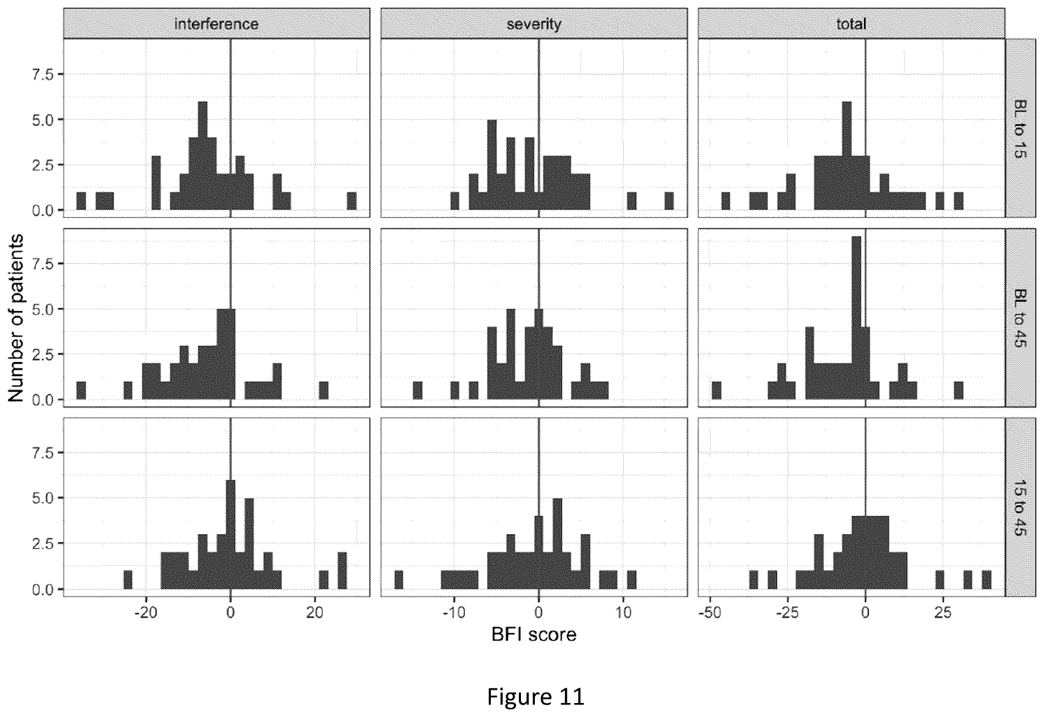

FIGS. 10 and 11 and the following tables describe summary statistics for changes in BFI.

TABLE 46
Summary statistics for change in BFI.

| | | | | | | |
|---|---|---|---|---|---|---|
| n | Change | Mean | SD | CI. low | CI. high | p. value |

Interference

| | | | | | | |
|---|---|---|---|---|---|---|
| NA | BL to 15 | −5.5 | 12.2 | −9.6 | −1.4 | 0.0105 |
| NA | BL to 45 | −5.8 | 10.9 | −9.5 | −2.1 | 0.0030 |
| NA | 15 to 45 | −0.3 | 11.0 | −4.0 | 3.4 | 0.8681 |

Severity

| | | | | | | |
|---|---|---|---|---|---|---|
| NA | BL to 15 | −0.6 | 5.3 | −2.4 | 1.2 | 0.4750 |
| NA | BL to 45 | −1.2 | 4.5 | −2.8 | 0.3 | 0.1064 |
| NA | 15 to 45 | −0.6 | 5.7 | −2.5 | 1.3 | 0.5259 |

Total BFI

| | | | | | | |
|---|---|---|---|---|---|---|
| NA | BL to 15 | −6.1 | 15.9 | −11.5 | −0.8 | 0.0267 |
| NA | BL to 45 | −7.1 | 14.3 | −11.9 | −2.2 | 0.0054 |
| NA | 15 to 45 | −0.9 | 14.9 | −5.9 | 4.1 | 0.7137 |

The following tables describe changes in mean scores using linear mixed models for the indicated outcome variables.

TABLE 47
Outcome variable: Interference.

| | est. | lower | upper | p. value |
|---|---|---|---|---|
| BL | 31.8 | 26.8 | 36.7 | <0.0001 |
| Change to 15 | −5.5 | −9.3 | −1.7 | 0.0050 |
| Change to 45 | −5.8 | −9.6 | −2.0 | 0.0031 |

Interference: Between-person SD at baseline is 14.1, so the mean changes are 39% & 41% of an SD.

TABLE 48
Outcome variable: Interference.

| | est. | lower | upper | p. value |
|---|---|---|---|---|
| Change to 15 | −39 | −66 | −12 | 0.0050 |
| Change to 45 | −41 | −68 | −14 | 0.0031 |

Interference: changes as percentages of baseline SD of 14.1.

TABLE 49
Outcome variable: Interference.

| Comparison | Correlation |
|---|---|
| BL and 15 | 0.64 |
| BL and 45 | 0.74 |
| 15 and 45 | 0.74 |

Correlations for interference.

TABLE 47
Outcome variable: Severity.

| | est. | lower | upper | p. value |
|---|---|---|---|---|
| BL | 17.1 | 15.0 | 19.1 | <0.0001 |
| Change to 15 | −0.6 | −2.4 | 1.1 | 0.4643 |
| Change to 45 | −1.3 | −3.0 | 0.5 | 0.1544 |

Severity: Between-person SD at baseline is 5.6, so the mean changes are 11% & 22% of an SD.

TABLE 48
Outcome variable: Severity.

| | est. | lower | upper | p. value |
|---|---|---|---|---|
| Change to 15 | −11 | −42 | 20 | 0.4643 |
| Change to 45 | −22 | −53 | 9 | 0.1544 |

Severity: changes as percentages of baseline SD of 5.6.

TABLE 49
Outcome variable: Severity.

| Comparison | Correlation |
|---|---|
| BL and 15 | 0.54 |
| BL and 45 | 0.80 |
| 15 and 45 | 0.65 |

Correlations for severity.

TABLE 50
Outcome variable: Total.

| | est. | lower | upper | p. value |
|---|---|---|---|---|
| BL | 48.8 | 42.1 | 55.6 | <0.0001 |
| Change to 15 | −6.1 | −11.1 | −1.1 | 0.0168 |
| Change to 45 | −7.1 | −12.1 | −2.1 | 0.0063 |

Total: Between-person SD at baseline is 19, so the mean changes are 32% & 37% of an S.

TABLE 51
Outcome variable: Total

| | est. | lower | upper | p. value |
|---|---|---|---|---|
| Change to 15 | −32 | −59 | 6 | 0.0168 |
| Change to 45 | −37 | | −11 | 0.0063 |

Total: changes as percentages of baseline SD of 19.

TABLE 52
Outcome variable: Total.

| Comparison | Correlation |
|---|---|
| BL and 15 | 0.66 |
| BL and 45 | 0.78 |
| 15 and 45 | 0.76 |

Correlations for total.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of improving cognition in a subject in need thereof comprising inhaling air having an elevated carbon dioxide concentration with a sequential rebreathing device.

2. The method of claim 1, wherein the sequential rebreathing device uses a breathing circuit that stores and then redirects the subject's exhaled gas back to the subject for inhalation following the inhalation of a small volume of oxygen containing an above atmospheric concentration, and a volume which is less than that required to maintain normal ventilation.

3. The method of claim 1, wherein the sequential rebreathing device provides at least about 0.5, 1.0, 1.5, 2.0, 2.5, 3.0, 3.5, 4.0, 4.5, 5.0, 5.5, or 6.0 liters per minute of oxygen.

4. The method of claim 1, wherein inhaling air having an elevated carbon dioxide concentration results in a PaCO₂ of 35 mmHg, 36 mmHg, 37 mmHg, 38 mmHg, 39 mmHg, 40 mmHg, 41 mmHg, 42 mmHg, 43 mmHg, 44 mmHg, 45 mmHg, 46 mmHg, 47 mmHg, 48 mmHg, 49 mmHg, 50 mmHg, 51 mmHg, 52 mmHg, 53 mmHg, 54 mmHg, or 55 mmHg.

5. The method of claim 1, wherein the subject in need is assessed to exhibit cognitive dysfunction.

6. The method of claim 1, wherein the subject in need thereof has, had, is suspected of having, or has been diagnosed with COVID, post-COVID conditions, post-COVID syndrome, long COVID, viral infection, post viral infection, bacterial infection, post bacterial infection, brain inflammation, mild cognitive impairment (MCI), Alzheimer's disease, early onset Alzheimer's disease, dementia, Lewey Body dementia, stroke, transient ischemic attack (TIA), head trauma, concussion, traumatic brain injury (TBI), or age-related cognitive decline.

7. The method of claim 1, wherein inhaling air having an elevated carbon dioxide concentration is performed for at least one period of at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, or at least about 60 minutes.

8. The method of claim 1, wherein inhaling air having an elevated carbon dioxide concentration is performed at least once per day, at least twice per day, at least three times per day, at least four times per day, at least five times per day, or at least six times per day.

9. The method of claim 1, wherein inhaling air having an elevated carbon dioxide concentration leads to increased blood flow in the brain.

10. The method of claim 1, wherein inhaling air having an elevated carbon dioxide concentration leads to reduced inflammation in the brain.

11. The method of claim 1, wherein inhaling air having an elevated carbon dioxide concentration leads to increased levels of serotonin in the brain.

12. The method of claim 1, wherein cognition is assessed at least one time before treatment.

13. The method of claim 1, wherein cognition is assessed at least one time after treatment.

14. The method of claim 1, wherein cognition is assessed using at least one cognitive assessment selected from the group consisting of Self-Administered Gerocognitive Exam (SAGE), Montreal Cognitive Assessment (MoCA), Mini-Mental State Exam (MMSE), Mini-Cog, Memory Impairment Screen (MIS), MIS by telephone (MIS-T), Mental Status Questionnaire (MSQ), 8-item Informant Interview (AD8), Functional Activities Questionnaire (FAQ), 7-Minute Screen (7 MS), Abbreviated Mental Test (AMT), St. Louis University Mental Status Examination (SLUMS), Telephone Instrument for Cognitive Status (TICS), TestMyBrain neurocognitive toolkit, Short Form 36 (SF-36) questionnaire, Brain Fog Questionnaire, and Informant Questionnaire on Cognitive Decline in the Elderly (IQCODE).

15. The method of claim 1, wherein cognition is improved following treatment when the cognitive assessment score determined after treatment is higher than the cognitive assessment score determined before treatment by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, or at least about 150%.
